(19) United States
(12) Plant Patent Application Publication
(10) Pub. No.: US 2003/0159187 P1
(43) Pub. Date: Aug. 21, 2003

(54) KIWI PLANT NAMED 'HORTGEM TORU'

(76) Inventors: Mark A. McNeilage, Auckland (NZ); Ron A. Beatson, Motueka (NZ); Elspeth A. MacRae, Auckland (NZ)

Correspondence Address: GREENLEE WINNER AND SULLIVAN P C, 5370 MANHATTAN CIRCLE, SUITE 201, BOULDER, CO, 80303, US

(21) Appl. No.: 10/079,142

(22) Filed: Feb. 19, 2002

(51) Int. Cl. A01H 5/00

(52) U.S. Cl. PLT/156

(57) ABSTRACT

A new and distinct kiwi plant of the species Actinidia arguta (Sieb. & Zucc.) Planch. ex Miq. var. arguta is described. The variety results from a controlled pollination using a female A. arguta selection AA01_{—}01 of unknown parentage and a male A. arguta selection AA04_{—}01 of unknown parentage. Both named parents (AA01_{—}01 and AA04_{—}01) are unpatented. The new variety is distinguished by its green hairless, edible skin, small fruit size, ovoid shape and sweet aromatic taste.

### BACKGROUND TO THE INVENTION

Kiwi plants in cultivation are mainly varieties of Actinidia deliciosa, particularly ‘Hayward’ although some A. chinensis and A. arguta varieties are grown. A. deliciosa and A. chinensis are closely related, whereas A. arguta is classified in a separate section of the genus. A. deliciosa and A. chinensis varieties have large fruit (˜100 g) with hair on the skin. The main varieties in New Zealand are ‘Hayward’ (A. deliciosa) (unpatented) and ‘Hort16A’ (A. chinensis) (PP11,066). Fruit are usually cut and eaten with a spoon. A. arguta has small fruit (˜10 g) with no hair on the skin. The skin is edible so these fruit can be eaten whole, like a grape.

All Actinidia species are dioecious, so female varieties have to be interplanted with male pollinizers to ensure fruit production.

A. arguta vines are deciduous and tend to grow vigorously in spring and summer when rapidly-growing shoots can intertwine and tangle if not managed. Vines do best in a mild temperate climate without late spring or early autumn frosts. They produce consistent heavy crops when grown in well-drained fertile soils and given regular irrigation in dry spells.

A. arguta flowers in spring (late October-early December) in New Zealand. Harvest of A. arguta fruit may occur between early February and late March in New Zealand depending on the selection and location of plantings. Compared to A. deliciosa and A. chinensis, A. arguta fruit require more careful handling during harvest and post-harvest procedures.

### SUMMARY OF THE INVENTION

The present invention is a new and distinctive kiwi plant having a small, generally ovoid shaped fruit with green hairless edible skin. This new variety is designated ‘Hortgem Toru’ and is derived from a controlled pollination of AA01_{—}01, a female A. arguta selection of unknown parentage (unpatented), with AA04_{—}01, a male A. arguta selection of unknown parentage (unpatented).

The female parent was introduced as a plant from Hillier's Nursery, England to New Zealand in 1955. The male parent arose from a seed family collected in the district Aomori, North Honshu, Japan and was introduced to New Zealand in 1977. The provenance of both is unknown.

This new variety was created during the course of a planned plant-breeding program, which was initiated during 1987 at HortResearch in Auckland, New Zealand. The controlled cross was made in November 1987. Seeds were sown in autumn (March) 1988 and 6 seedlings were selected from this cross and were planted out in the field at HortResearch Kumeu Research Orchard in spring (October) 1988. The seedlings first fruited in February-March 1991. Twenty promising female seedlings were clonally propagated into a two-site replicated trial in 1995 and ‘Hortgem Toru’ (breeding code C3C3) was selected after storage and sensory evaluation in 1998.

The new variety can be asexually reproduced as cuttings or by grafting or budding on to seedling or cutting-grown rootstocks of A. arguta. Trial plantings as cuttings established in 1995 at the HortResearch Te Puke and Nelson Research Centres and on seedling rootstocks established in 1998 at these sites have shown that the unique combination of characters come true to form and are established and transmitted through succeeding asexual propagations.

### BRIEF DESCRIPTION OF THE ILLUSTRATIONS

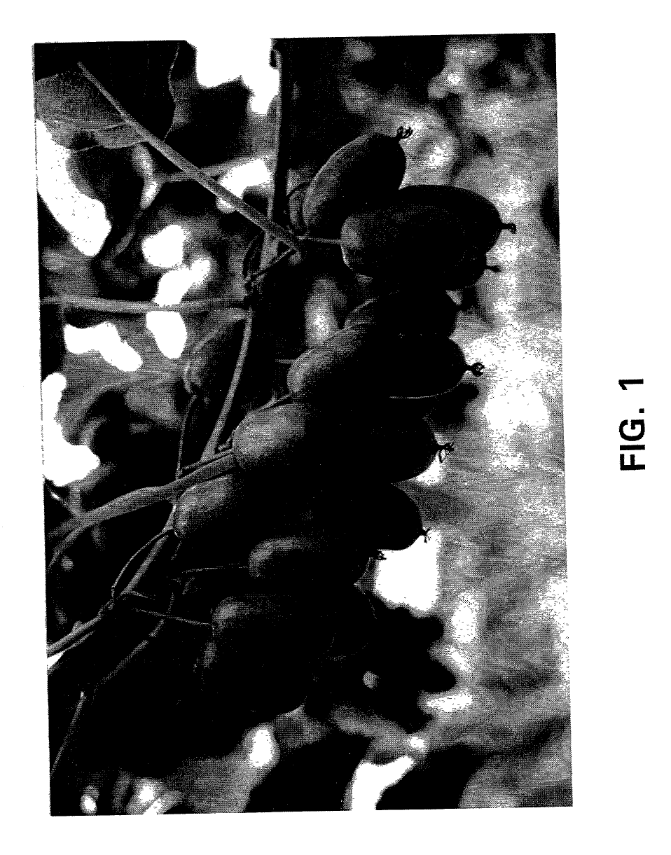

FIG. 1 shows typical fruit of the variety ‘Hortgem Toru’ in the orchard

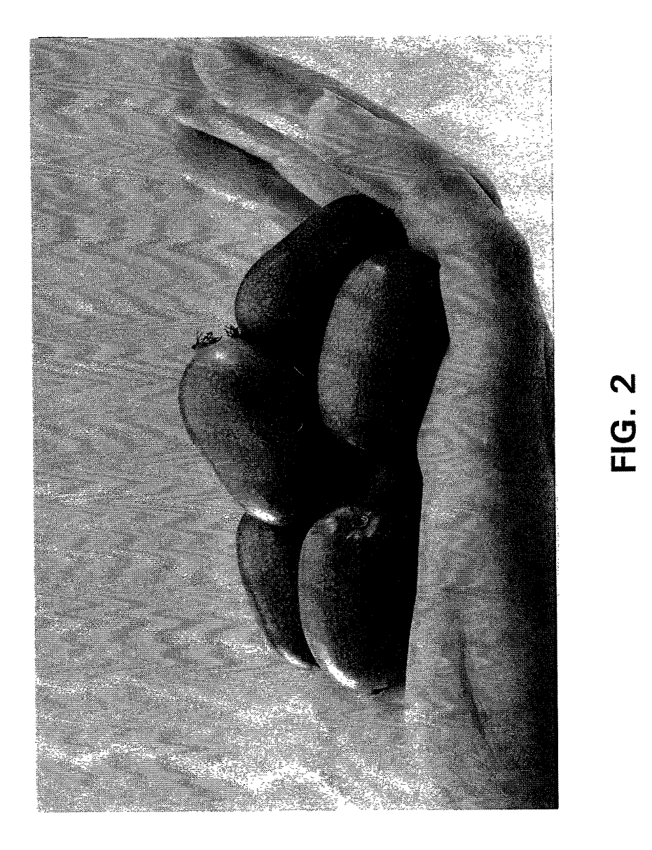

FIG. 2 shows typical fruit of the variety ‘Hortgem Toru’ in the studio

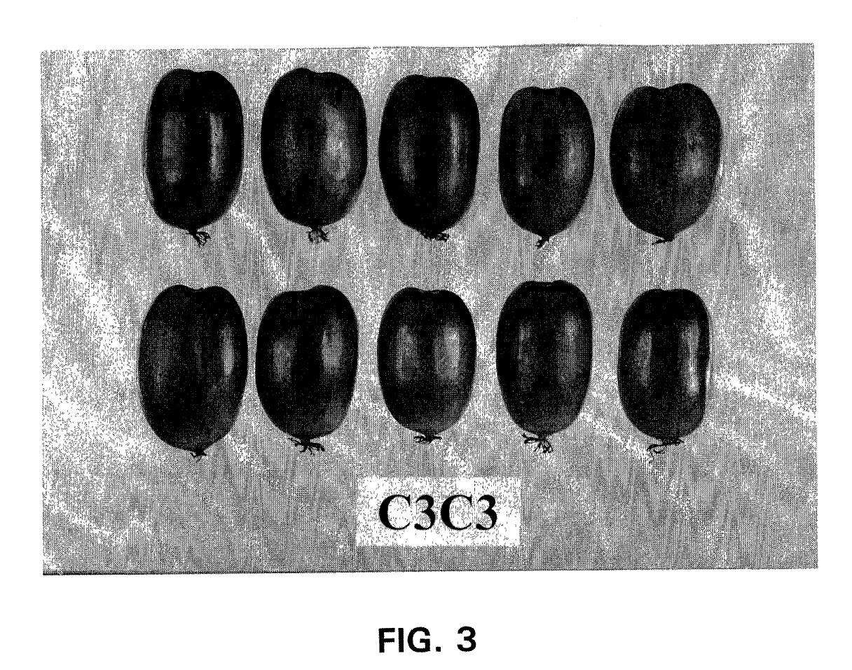

FIG. 3 shows the side view of typical fruit of the variety ‘Hortgem Toru’

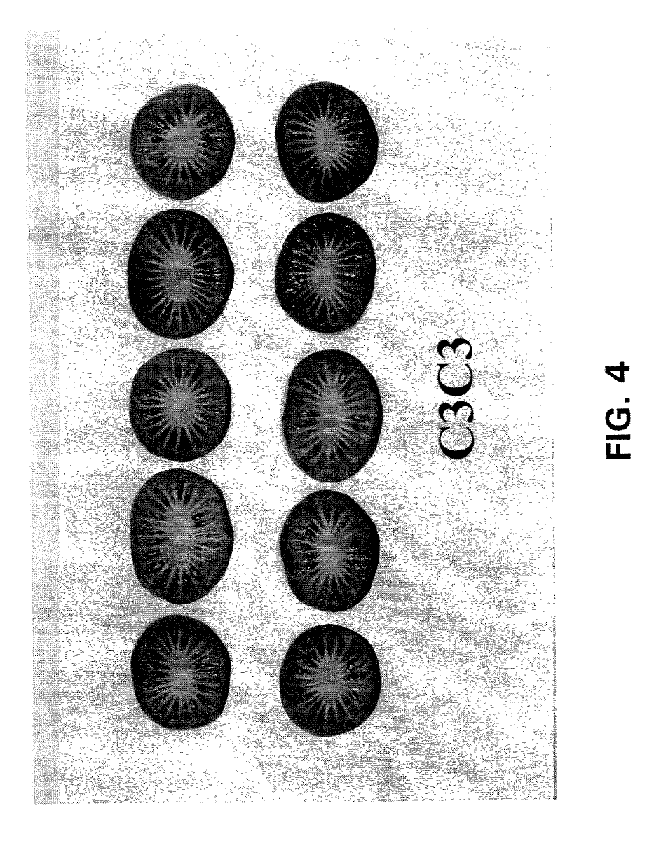

FIG. 4 shows the cross-sectional view of typical fruit of the variety ‘Hortgem Toru’

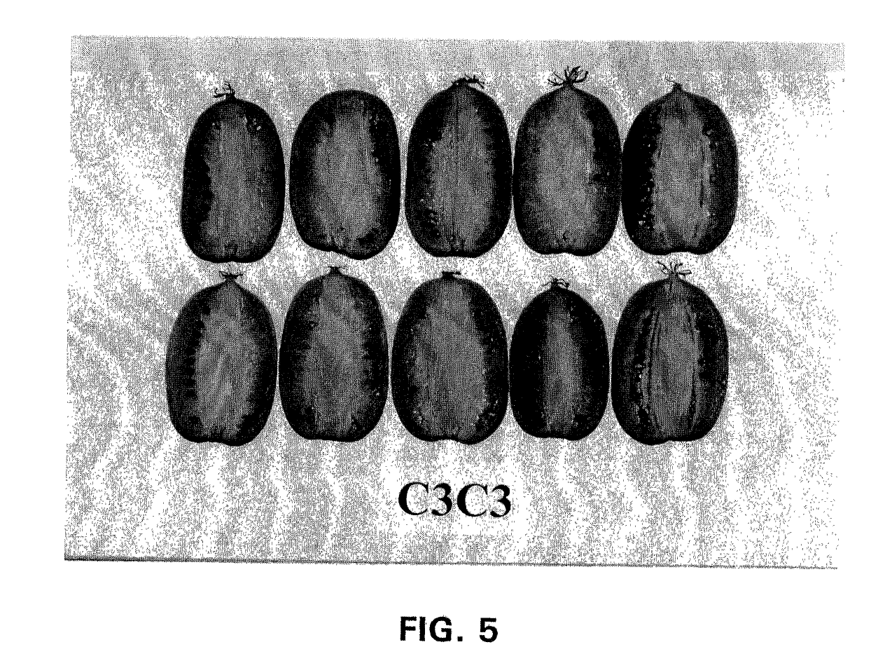

FIG. 5 shows the longitudinal view of typical fruit of the variety ‘Hortgem Toru’

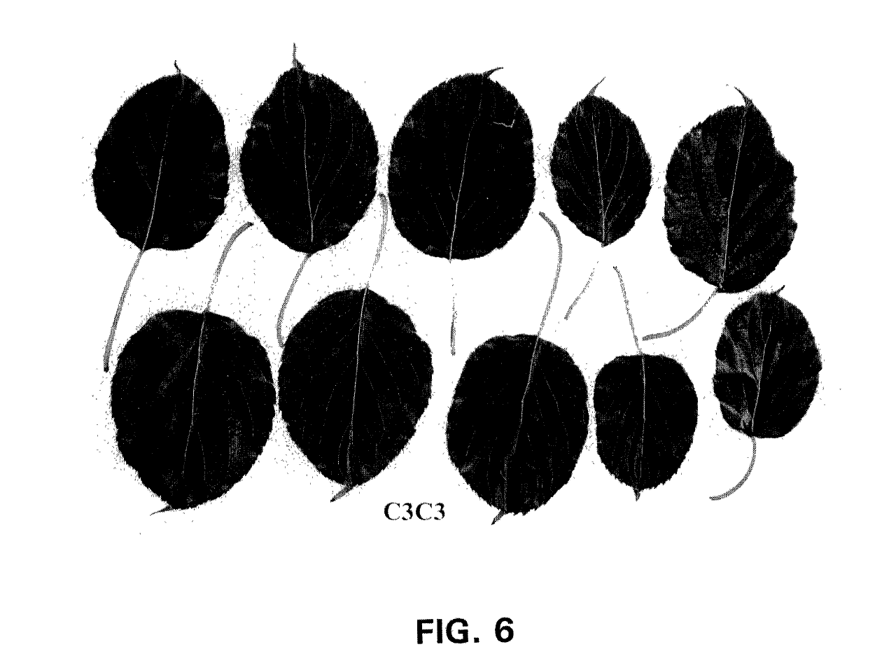

FIG. 6 shows leaves of the variety ‘Hortgem Toru’

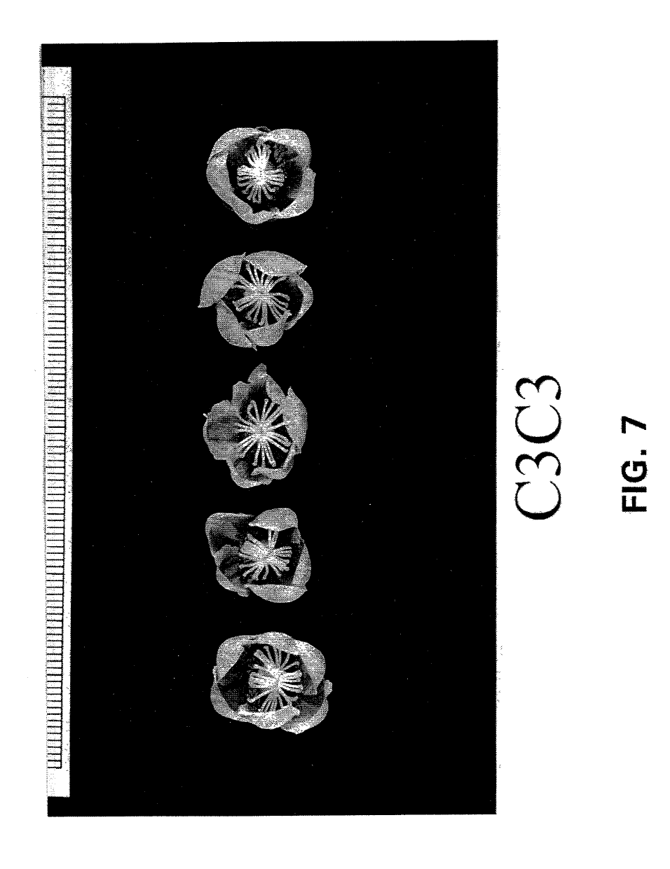

FIG. 7 shows flowers of the variety ‘Hortgem Toru’

Photographs of fruit were taken after the normal harvest date and are depicted in colours as nearly true as is reasonably possible to make the same in a colour illustration of this character. Fruit skin colour may vary depending upon extent of exposure to direct sunlight.

### COMPARISON TO CLOSEST VARIETY

The distinctive characteristics of this new kiwi variety, described in detail below, were observed in 2001 at Te Puke, New Zealand. The plants observed were established from cuttings and were 6 years old at the time. ‘Hortgem Toru’ is a new variety of A. arguta distinctive in its own right. Comparison with another similar variety ‘Hortgem Tahi’ (application Ser. No. 09/780,809) showed that ‘Hortgem Toru’ may be distinguished as follows:

| | | Expression | Expression of |
|---|---|---|---|
| | | of the | the character- |
| | | characteristic | ristic for |
| | | for the similar | the candidate |
| Similar variety | Characteristic | variety | variety |
| Hortgem Tahi | Fruit shape | spheroid | ovoid |
| Hortgem Tahi | Start of harvest | late Feb | early Feb |
| Hortgem Tahi | Storage life of fruit at | 10-12 weeks | 3-4 weeks |
| | 0° C. in air storage | | |

### BOTANICAL DESCRIPTION OF THE PLANT

The new variety ‘Hortgem Toru’ is pistallate, with morphologically perfect but functionally imperfect flowers, i.e. the flowers produce only sterile pollen and thus require a pollinizer for fruit production. Characteristics of the new variety include a short period (˜3 months) between flowering and harvest, early harvest, small ovoid fruit with green hairless edible skin, green flesh and a sweet aromatic flavour.

### TABLE OF CHARACTERISTICS

Horticultural terminology is used in accordance with revised UPOV guidelines for kiwi. All dimensions in millimetres, weights in grams (unless otherwise stated). Where a colour reference is given these refer to the RHS Colour Chart, The Royal Horticultural Society, London. 3^{rd} Edition, 1995.

Plant:

(Measurements from samples of 20, unless stated)

Plant: sex expression.—Female.

Plant: vigour.—Strong.

Young Shoot:

Young shoot: hairs.—Present.

Young shoot: density of hairs.—Medium.

Young shoot: type of hairs.—Tomentose.

Young shoot: anthocyanin coloration of growing tip.—Medium.

Young shoot: anthocyanin coloration of leaf axil.—Absent or very weak.

Stem:

Stem: coloration of leaf axil.—Absent or very weak.

Stem: diameter.—Thin; mean 8.4.

Stem: dormant bud diameter.—Very small: 0.2.

Stem: color on upper side of shoot.—Greyed-Orange 166B.

Stem: character of bark.—Smooth.

Stem: hairs.—Absent.

Stem: conspicuousness of lenticels.—Conspicuous.

Stem: number of lenticels.—Many.

Stem: color of lenticels.—Light red brown.

Stem: size of bud support.—4.0.

Stem: visibility of bud (dormant canes).—Visible.

Stem: number of hairs visible on bud (dormant canes).—Absent.

Stem: leaf scar.—Deep.

Leaf (Mature):

Leaf: general shape of blade.—Broad ovate.

Leaf: length.—114.7 mm (97-134).

Leaf: width.—79.2 mm (67-90).

Leaf: petiole length.—60.2 (46-85).

Leaf: shape of tip of blade.—Caudate.

Leaf: shape of base of blade.—Rounded.

Leaf: arrangement of leaf bases.—Far apart.

Leaf: puckering/blistering on upper side of blade.—Absent or very weak.

Leaf: margin.—Ciliate.

Leaf: green color of upper side of blade.—Green 136A.

Leaf: glossiness of upper surface of blade.—Medium.

Leaf: color of lower side of blade.—Green 138A.

Leaf: glaucosity (lower side of blade).—Absent.

Leaf: hairs on petiole.—Absent or very weak.

Leaf: density of hairs on petiole.—Absent or very sparse.

Leaf: anthocyanin coloration on upper side of petiole.—Medium.

Flower:

Inflorescence: predominant number of flowers.—Three.

Pedicel: length.—31.0 (range 28.6-33.6).

Pedicel: hairs.—Sparse.

Pedicel: length of hairs.—Very short.

Flower: number of sepals.—5.

Flower: color of sepals.—Reddish brown.

Flower: diameter (terminal or king flower when fully open).—Small 23.6 (range 20.1-26.4).

Flower: petal length.—12.4 (range 11.1-13.6).

Flower: petal width.—10.1 (range 8.1-12.1).

Flower: petal length/width ratio.—1.2 (range 1.02-1.53).

Flower: mean number of petals per flower.—5.4 (range 5-6).

Flower: number of flowers with more than six petals.—0 out of 20 (12 with 5, 8 with 6).

Flower: arrangement of petals.—Overlapping.

Flower: petal shoulder.—Non-constrict.

Flower: primary color of petals (when fully open).—Green white 157A.

Flower: type of coloration of petals.—Uniform over entire petal.

Flower: number of stamens.—39.7 (range 38-42).

Flower: length of stamen filaments.—3.5 (range 3.0-4.1).

Flower: length of anthers.—2.2 (range 1.5-2.8).

Flower: filament color.—Light green.

Flower: anther color.—Dark purple.

Flower: style number.—23.0 (range 20-29).

Flower: length of styles.—4.8 (range 4.3-5.4).

Flower: attitude of styles.—Most are horizontal.

Flower: curvature of styles.—Weak.

Flower: length of ovary.—7.3 (range 7.0-7.9).

Flower: amount of hair on ovary.—Absent.

Flower: colour of ovary.—Yellow green 145A.

Fruit:

Fruit: overall size.—11.85 (range 9.66-13.57).

Fruit: length.—34.8 (33-37).

Fruit: width (max).—21.6 (20-23).

Fruit: width (min).—19.6 (18-21).

Fruit: core diameter (max).—6.7 (5.2-9.3).

Fruit: core diameter (min).—4.7 (3.9-5.4).

Fruit: locule number.—20.7 (18-24).

Fruit: peduncle length.—31.1 (28.3-34.0).

Fruit: peduncle width.—1.5 (1.3-2.0).

Fruit: general shape.—Oblong.

Fruit: cross section at median.—Oblate.

Fruit: general shape of stylar end.—Slightly pointed protruding.

Fruit: shape of shoulder on stalk end.—Rounded.

Fruit: skin color at harvest (fruit still hard).—Medium green.

Fruit: skin colour change during ripening.—Medium green.

Fruit: skin color at maturity for consumption.—Medium green.

Fruit: hairs.—absent.

Fruit: core diameter (at largest diameter).—Medium.

Fruit: core shape (in cross section).—Oblate.

Fruit: core woody spike.—Absent.

Fruit: outer pericarp color at maturity for consumption.—Green.

Fruit: inner pericarp col. (locules) at mat. for consumption .—Green.

Fruit: core color at maturity.—Green white.

Fruit: seed colour at maturity (in flesh).—Dark red brown.

Fruit: seed colour when dry.—Red brown.

Events:

Time of vegetative budbreak.—Late August-early September.

Time of beginning of flowering.—Late October-early November.

Time of maturity for harvest (at nominated Brix level).—Early February.

### HORTICULTURAL CHARACTERISTICS

Details below relate to observations made on cutting-grown plants growing at HortResearch Te Puke Research Centre, New Zealand. These vines were 3 years old.

Cropping.—young vines of ‘Hortgem Toru’ are precocious, beginning to bear in their second year and are expected to reach full capacity at about 7 years. The storage life of ‘Hortgem Toru’ fruit is 3-4 weeks at 0° C., if stored in unventilated containers in air storage.

Yield and fruit size.—(Data from harvesting all fruit from 6 vines in early February 1999).

Mean fruit weight.—11.85 g Maximum: 13.57 g Minimum: 9.66 g.

Mean fruit number.—356 Maximum: 922 Minimum: 2.

Mean yield.—4.2 kg Maximum: 10.7 kg Minimum: 0.027 kg.

It is anticipated mature, well-managed vines would yield approximately 5000 fruit per vine with a mean fruit weight of 10 g and mean yield per vine of 50 kg.

We claim:

1. A new and distinct kiwi plant of the species A. arguta substantially as described and illustrated, characterised by small ovoid fruit with green hairless edible skin and green flesh with a sweet aromatic flavour.
